(19) United States
(12) Patent Application Publication
(10) Pub. No.: US 2014/0322078 A1
(43) Pub. Date: Oct. 30, 2014

(54) METHOD FOR DECONTAMINATING BRICK OR CONCRETE

(71) Applicant: Institute of Nuclear Energy Research, Atomic Energy Council, Executive Yuan, R.O.C., (US)

(72) Inventors: Ming-Tsai Liang, Kaohsiung (TW); Chih-Hsiung Lin, Kaohsiung (TW); Yuh-Wern Wu, Kaohsiung (TW); Chin-Hsiang Kan, Hsinchu County (TW); Wen-Cheng Lee, Taoyuan County (TW)

(73) Assignee: Institute of Nuclear Energy Research, Atomic Energy Council, Executive Yuan, R.O.C., Taoyuan County (TW)

(21) Appl. No.: 13/873,702

(22) Filed: Apr. 30, 2013

(51) Int. Cl. A61L 2/18

(52) U.S. Cl. CPC A61L 2/18
USPC 422/32

(57) ABSTRACT

A method for decontaminating a brick or concrete according to the present invention comprises the steps of: pulverizing the brick or the concrete by using a pulverizing device (pulverizing step); arranging the brick or concrete in a container room and washing the brick or concrete by using a decontamination agent in the container room (washing), wherein the container room has a pressure of above 74 Pa, and a temperature of above 32° C., and the decontamination agent includes a supercritical carbon dioxide flow, a cosolvent and a metal chelating agent; introducing a replacing fluid to the container room to replace the decontamination agent in the container room and then separating the replacing fluid and the brick or concrete (replacing and separating step); and acid cleaning the cosolvent and the metal chelating agent by using an acidic solution (acid cleaning step). A radionuclide may be decontaminated from the brick or concrete.

### FIELD OF THE INVENTION

The present invention is related to a method for decontaminating a brick or concrete, particularly to a method for processing a radionuclide from a brick or concrete, so that the nuclide amount may be effectively reduced and the brick or concrete may be recycled or buried in a stable state.

### DESCRIPTION OF THE RELATED ART

With continuous development of industries and advancement of technologies, electricity demands are also going high. For the global energy resources, petrochemical fuels such as oil, coal, and natural gas are generally used. However, a huge amount of waste gas, air pollution, and carbon dioxide leads to the greenhouse effect, causing people to resort to nuclear electricity so as to satisfy the huge electricity demand. Nuclear electricity is purported to be safe and clean, but nuclear electricity events are still presented ever, such as Chernobyl and Fukashima nuclear events.

This Fukashima event happened on 2011 in Japan brought about the nuclear plant there in a prehistoric catastrophic state, surrounding soils and buildings endured bad pollution, and a plenty of surface soils and building debris, in which a large amount of radionuclide, such as strontium (Sr), is mixed, are required to be decontaminated and processed. For concrete pollution, its pollution extent is related to the pollution position and the involved metal type. In a deep pollution, a pollution depth of a few mili-meters to centimeters is possible.

Conventional concrete surface contaminating technologies include scabbling, shaving, slicing/sawing, and polishing/sand-blasting (wet polishing material). Detaching technologies include jackhammer, drilling/crashing and blasting.

Although the prior art has provided a solution for active carbon revival and repeated soil fostering, the objects of simple manufacturing process, low cost, few waste liquid and low energy consumption still haven't been met up with. In view of this, a method for processing radionuclide in active carbon, soil and building debris, such as a brick or concrete is a necessity in the industry.

In view of the drawbacks mentioned above, the inventor of the present invention provides a method for contaminating a brick or concrete, after many efforts and researches to overcome the shortcoming encountered in the prior art.

### SUMMARY OF THE DISCLOSURE

It is, therefore, a main object of the present invention to provide a method for decontaminating a brick or concrete so that radionuclide therein may be effectively decontaminated.

It is a secondary object of the present invention to provide a method for decontaminating a brick or concrete with a reduced amount of organic solvent and water consumption in the decontaminating process.

To achieve the above objects, the method for decontaminating a brick or concrete according to the present invention comprises the steps of: pulverizing the brick or the concrete by using a pulverizing device (pulverizing step); arranging the brick or concrete in a container room and washing the brick or concrete by using a decontamination agent in the container room (washing step), wherein the container room has a pressure of 200 to 350 Pa, and a temperature of 40 to 140° C., and the decontamination agent includes a supercritical carbon dioxide flow, a cosolvent and a metal chelating agent; introducing a replacing fluid to the container room to replace the decontamination agent in the same and then separating the replacing fluid and the brick or concrete (replacing/separating step); and acid cleaning the cosolvent and the metal chelating agent by using an acidic solution (acid cleaning step).

The metal chelating agent is selected from a group consisting of: ithiocarbamate dibutyldithiocarbamate (BDC), dipentyldithiocarbamate (P5DC), dihexyldithiocarbamate (P6DC) or pyrrolidine-dithiocarbamate, acetylacetone (AA), trifluoroacetylacetone (TFA), hexafluoroacetyl-acetone (HFA), thenoyltrifluoroacetone (TTA) or heptafluorobutanoyl-pivaroylmethane (FOD), tributylphosphate (TBP), tributylphosphine oxide (TBPO), trioctylphosphine oxide(TOPO), triphenylphosphine oxide (TPPO), bis(2,4,4,-trimethylpentyl) phosphinic acid (cyanex 272), bis(2,4,4,-trimethylpentyl) dithiophosphinic acid (Cyanex 301), bis(2,4,4,-trimethylpentyl) monothiophosphinic acid (cyanex 302), and di(2-ethylhexyl) phosphoric acid (D2EHPA) or crown ether.

Preferably, the metal chelating agent is di(2-ethylhexyl) phosphoric acid.

Preferably, the cosolvent is preferably low-carbon alkane or alcohol, having a number of carbon of 1 to 6, and more preferably methyl alcohol or n-hexane.

Preferably, the pressure of the container room is above 74 bar, more preferably 100 to 350 bar, and the temperature of the container room is above 32° C., and more preferably 35° C. to 140° C.

In the present invention, the cosolvent has a weight percentage of 1.0 to 20.0%, the metal chelating agent 0.1 to 9.0%, the supercritical carbon dioxide flow 79.9 to 98.9% in the decontamination agent, respectively.

The metal chelating agent is selected from a group consisting of: ithiocarbamate dibutyldithiocarbamate (BDC), dipentyldithiocarbamate (P5DC), dihexyldithiocarbamate (P6DC) or pyrrolidine-dithiocarbamate, acetylacetone (AA), trifluoroacetylacetone (TFA), hexafluoroacetyl-acetone (HFA), thenoyltrifluoroacetone (TTA) or heptafluorobutanoyl-pivaroylmethane (FOD), tributylphosphate (TBP), tributylphosphine oxide (TBPO), trioctylphosphine oxide (TOPO), triphenylphosphine oxide (TPPO), bis(2,4,4,-trimethylpentyl) phosphinic acid (cyanex 272), bis(2,4,4,-trimethylpentyl) dithiophosphinic acid (cyanex 301), bis(2,4,4,-trimethylpentyl) monothiophosphinic acid (cyanex 302), and di(2-ethylhexyl) phosphoric acid (D2EHPA) or crown ether.

Preferably, the metal chelating agent is D₂EHPA. Preferably, the cosolvent is preferably low-carbon alkane or alcohol, having a number of carbon of 1 to 6, and more preferably methyl alcohol or n-hexane.

### BRIEF DESCRIPTIONS OF THE DRAWINGS

The present invention will be better understood from the following detailed descriptions of the preferred embodiments according to the present invention, taken in conjunction with the accompanying drawings, in which:

FIG. 1 is a flowchart of a method for decontaminating a brick or concrete according to the present invention;

FIG. 2 is a schematic diagram of an arrangement of a supercritical flow device according to an embodiment of the present invention;

FIG. 3 is a plot showing a relationship of Sr metal removing rate v.s. time according to a first embodiment in the present invention; and

FIG. 4 is a plot showing a relationship of Sr metal removing rate v.s. time according to a second embodiment in the present invention.

### DESCRIPTION OF THE PREFERRED EMBODIMENTS

To enable the above and other objects, features and advantages to be readily understood, the preferable embodiments with the accompanying drawings are exemplified as the followings.

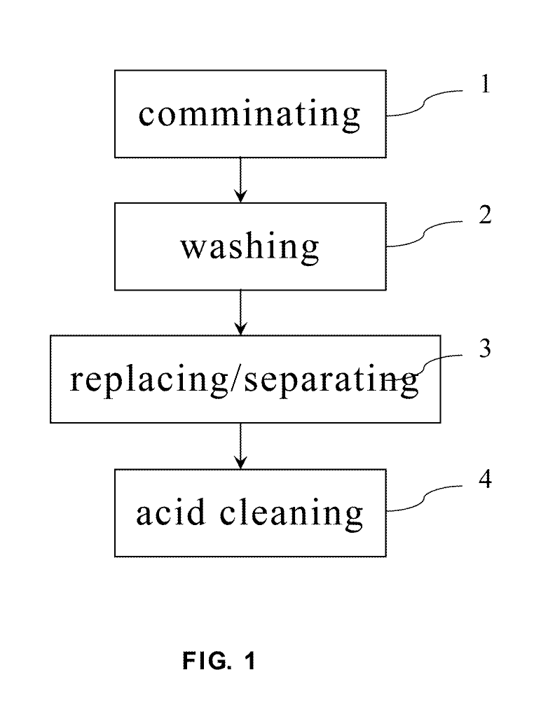

Referring to FIG. 1, a method for decontaminating a brick or concrete comprises the steps of pulverizing (1), washing (2), replacing/separating (3), and acid washing (4).

In step (1), a pulverizing device is used to comminute the brick or the concrete with metallic pollution, so that a load-in amount and a total contact area of the brick or the concrete are increased.

In step (2), the brick or concrete is arranged in a container room and a decontamination agent is introduced to wash the brick or concrete in the container room. The decontamination agent includes a supercritical carbon dioxide flow, a cosolvent and a metal chelating agent. The container room is given a pressure and temperature defined from the work pressure and temperature of the supercritical carbon dioxide flow, which are apparent to those who skilled in the art. In this embodiment, the pressure of the container room is above 74 bar, and more preferably 100 to 350 bar. The temperature of the container room is above 32° C., and more preferably 35° C. to 140° C. The container room has a pressure of 100 to 350 Pa and a temperature of 35 to 120° C.

More specifically, the cosolvent in the decontamination agent is good to dissolve the metal compound and provides an aid on dissolving a radionuclide in the brick or concrete into the decontamination agent under the conditions of the inner temperature and pressure in the container room. As such, the radionuclide may loose itself from the brick or concrete, and thus the containment of the radionuclide has a reduced amount in the brick or concrete.

For example, the auxiliary agent is used as a diluting solution for the metal chelating agent, and is a low-carbon alkane or alcohol, having a number of carbon of 1 to 6 (C1 to C6), and particularly methyl alcohol or hexane. The auxiliary agent not only has an assistance to promote an absorption result of the metal chelating agent, but also help changing a polarity characteristic of the supercritical carbon dioxide flow.

The metal chelating agent may be a metal chelate or ligand organic compound. The metal chelating agent may be classified into four types: (1) dithiocarbamate metal chelating agent, (2) diketone metal chelating agent, (3) organic phosphoric-based metal chelating agent, and (4) macrocycle metal compound.

The dithiocarbamate metal chelating agent includes bis-trifluoroethyldithiocarbamate (FDDC), diethyl-dithiocarbamate (DDC), dipropyldithiocarbamate (P3DC), Dibutyldithiocarbamate (BDC), dipentyldithiocarbamate (P5DC), dihexyldithiocarbamate (P6DC) and pyrrolidinedithiocarbamate (PDC).

The diketone-based metal chelating agent includes acetylacetone (AA), trifluoroacetylacetone (TFA), hexafluoroacetylacetone (HFA), thenoyltrifluoroacetone (TTA) and heptafluorobutanoyl-pivaroylmethane (2 FOD).

An organic metal chelating agent includes tributylphosphate (TBP), tributylphosphine oxide (TBPO), trioctylphosphine oxide (TOPO), triphenylphosphine oxide (TPPO), bis(2,4,4,-trimethylpentyl)phosphinic acid (cyanex 272), bis(2,4,4,-trimethylpentyl)dithio-phosphinic acid (cyanex 301), bis(2,4,4,-trimethyl-pentyl)monothiophosphinic acid (cyanex 302) and di(2-ethylhexyl)phosphoric acid (D2EHPA).

A macrocycle metal chelating agent includes crown ether.

In this embodiment, D2EHPA is selected, which can have an effect on strontium (Sr) metal in the brick or concrete along with the supercritical carbon dioxide flow and the cosolvent. This is because carbon dioxide is easy to be acquired, and without color, flavor, toxicity, explosive characteristic, erosive characteristic flammability, and thus safe to use.

In addition, the supercritical carbon dioxide flow is good to uniformly contact with the brick or concrete. Further, in virtue of the polarity effect of the supercritical carbon dioxide flow, the radionuclide is released. Further, the metal chelating agent seizes and dissolved the released radionuclide into the decontamination agent. As such, the inventive decontaminating method has the effect between the metal compound and the supercritical carbon dioxide and thus enhances a combination efficiency of Sr metal in the brick or concrete.

In replacement/separation step (2), a replacing fluid is introduced into the container room to replace the decontamination agent within the container room, so as to separate the replacing fluid with the brick or concrete. More specifically, a supercritical flow may be selected as the replacing fluid. In this embodiment, the supercritical flow is used to replace the decontaminating agent within the container room. Next, the pressure within the container room is reduced so that the supercritical carbon dioxide converts to a gaseous state. The carbon dioxide is directly discharged to the ambient or recycled to be further use.

In this embodiment, the pressure and temperature within the container room are reduced to be the same with those in the ambient and the carbon dioxide is directly discharged outside the container room. At this time, the decontaminating process of the radionuclide from the brick or concrete within the container room is totally completed.

In this manner, the method for decontaminating a brick or concrete can effectively reduce a use amount of the organic solvent, save energy used to heat the brick or concrete and the decontamination agent, and clean the brick or concrete without requiring a large amount of aqueous. In addition, no organic solvent is left in the processed brick or concrete, and the radionuclide remaining in the brick or concrete is well reduced.

Now, an experiment is executed for proof of the effectiveness of the reduction of the radionuclide in the brick or concrete. In this embodiment, a set of 3 kilograms of pulverized brick and concrete is soaked in a strontium nitrate solution (30 g/L) of 5 liters for 48 hr., respectively. Then, the brick or concrete is dried to form a simulated metal-polluted brick or concrete. Then, the following experiment objects are performed: (A) analysis of Sr metal containment in the brick or concrete, (B) analysis of Sr metal containment in the cosolvent and the metal chelating agent, (C) removing of the Sr metal from the pulverized brick, and (D) removing of the Sr metal from the pulverized concrete.

(A) Analysis of Sr metal containment in the brick or concrete:

In this embodiment, this analysis is performed by using an Inductively Coupled Plasma-Optical Emission Spectrometer (ICP-OES), and described as follows:

1. put a 0.1 g (Ws) brick or concrete within a microwave digestion bin;

2. subsequently introduce 4.5 mL of thick nitrate and 1.5 mL of think hydrochloric;

3. put free of the microwave digestion bin for 20 min. until the end of the reaction, and then the digestion bin is put into a microwave digestion means;

4. set a microwave temperature at 220° C. for 20 min., a temperature rising time as 30 min., and a temperature maintain time as 50 min.

5. put the digestion bin into a water bath for 20 min., and then adjust the digestion liquid to be 25 mL by using a deionized water;

6. filter out the digestion liquid and introduce the filtered digestion liquid into an empty test tube with a seal sheet sealing on an opening of the test tube, for the following analysis.

7. multiply the obtained Sr metal concentration Cs with the volume 0.025 L and the weight of the cleaned brick or concrete W_{ST} loaded into the container room (loaded-in pulverized brick 280 g and loaded-in pulverized concrete 370 g), and the obtained value is divided by the weight of the brick or concrete W_{S} in the digestion bin. The result shows the weight of the Sr metal remaining in the brick or concrete W_{SSr}=C_{S}×0.025×W_{ST}/W_{S}.

In this embodiment, the Sr metal containment in per gram of the metal-polluted pulverized brick is about 2.08 mg, while the Sr metal containment in per gram of the metal polluted pulverized concrete is about 8.33 mg.

(B) Analysis of Sr metal containment in the cosolvent and metal chelating agent:

In this embodiment, this analysis is performed by using an Inductively Coupled Plasma-Optical Emission Spectrometer (ICP-OES), and described as follows:

1. measure the weight of the cosolvent and metal chelating agent collected at different time points, respectively, and deduct each of such weights with a weight an empty bin to obtain a real weight W_{E};

2. extract 1 mL of such liquid, and arrange the extracted liquid within a 60° C. oven to eliminate the cosolvent, then introduce 0.5 mL n-Hexane, then 10% nitrate aqueous solution 10 mL, subject the resulting solution to an ultrasonic vibration for 10 min. and then let it in a rest state for 16 hr., decontaminate an upper layer of liquid while retain the rest portion of the extracted liquid, and deduce a Sr metal concentration C_{E} by using the ICP analysis.

3. obtaining the weight of the Sr metal W_{ESr} by multiplying the volume 0.01 L of the extracted liquid with the Sr metal concentration C_{E} and V_{E}, as the following equation:

W_{ESr}=C_{E}×0.01×V_{E}.

(C) Sr metal reduction experiment with respect to a pulverized brick sample:

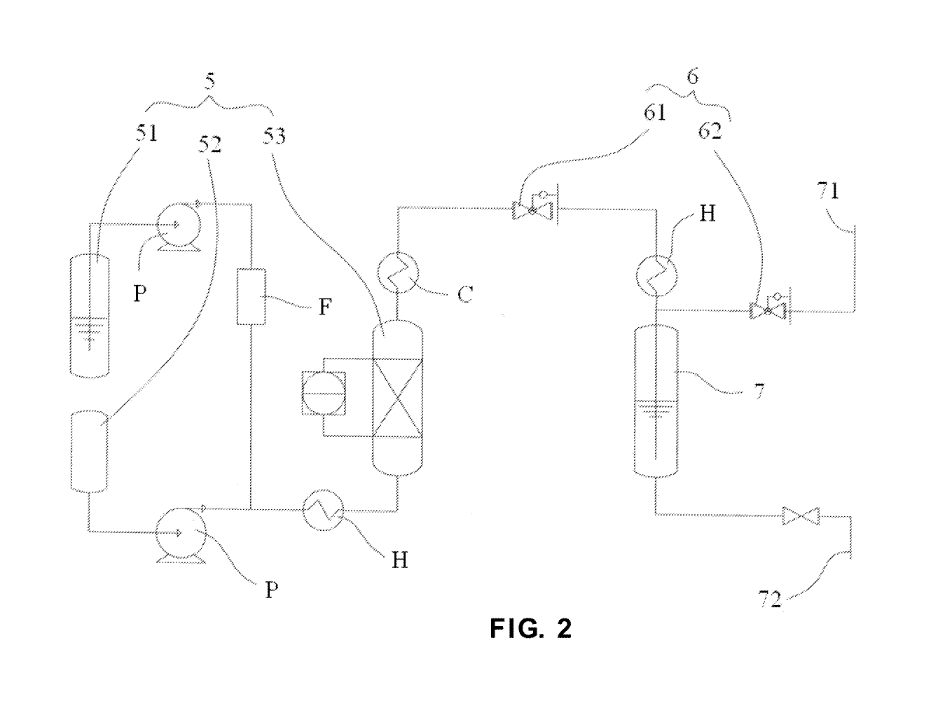

This embodiment provides a supercritical flow device as shown in FIG. 2, comprises a hybrid assembly 5, a pressure assembly 6 and a separation trough 7. The hybrid assembly 5 is connected and communicated to the separation trough 7, and the pressure assembly 6 is used to control an inner pressure of the hybrid assembly 5 and the separation trough 7.

The hybrid assembly 5 has a carbon dioxide trough 51, a cosolvent and a metal compound trough 52 and a container room 53. The carbon dioxide trough 51 and the cosolvent and metal chelating agent trough 52 are each connected and communicated to the container room 53. The carbon dioxide trough 51 is used to receive carbon dioxide inside. The cosolvent and metal chelating agent trough 52 is used to receive the cosolvent (e.g. methyl alcohol or n-Hexane) and the metal chelating agent (e.g. D₂EHPA) inside. The container room 53 is used to receive a to-be-processed sample. The carbon dioixide trough 51 and the cosolvent and metal chelating agent trough 52 is provided with an adjustment function for the inner temperature and pressure (such as the heater H or cooler C shown in figure), and each connected to a liquid delivery pump P and a flow regulator F, so that the solution inside can be delivered to the container room 53, respectively.

The pressure assembly 6 includes a first back pressure valve 61 and a second back pressure valve 62, for controlling the inner pressure within the container room 53 and the separation trough 7, respectively.

The separation trough 7 comprises a first exit 71 and a second exit 72. The first exit 71 provides an outlet for the gas inside releases to the ambient or a collect bin (now shown). The second exit 72 provides an outlet for the once Sr metal mixed cosolvent and the metal chelating agent.

In this embodiment, the supercritical flow device is operated as following description: put a brick or concrete sample into the container room 53, adjust the pressure and temperature of the container room 53 to their designated values, and re-adjust the operation flow into the container room 53 at a volume speed 4.5 mL/min (in a mixed state for the cosolvent and metal chelating agent). Then, 30 g/min. is set as a speed of a carbon dioxide flow washing for removing the radionuclide for a time, e.g. 3 to 6 hours. The operation flow flows from the container room 53 to the separation trough 7 to reduce the pressure of the separation trough 7 down to 40 bar, vaporizing the supercritical carbon dioxide, which may be recycled to use or discharged to the ambient. Now in the separation trough 7, an extracted solution of the cosolvent and metal chelating agent is left, which includes a metal chelating agent and the other metal chelating agent mixed with Sr metal. In the current container room 53, the brick or concrete has been the state with the Sr metal eliminated.

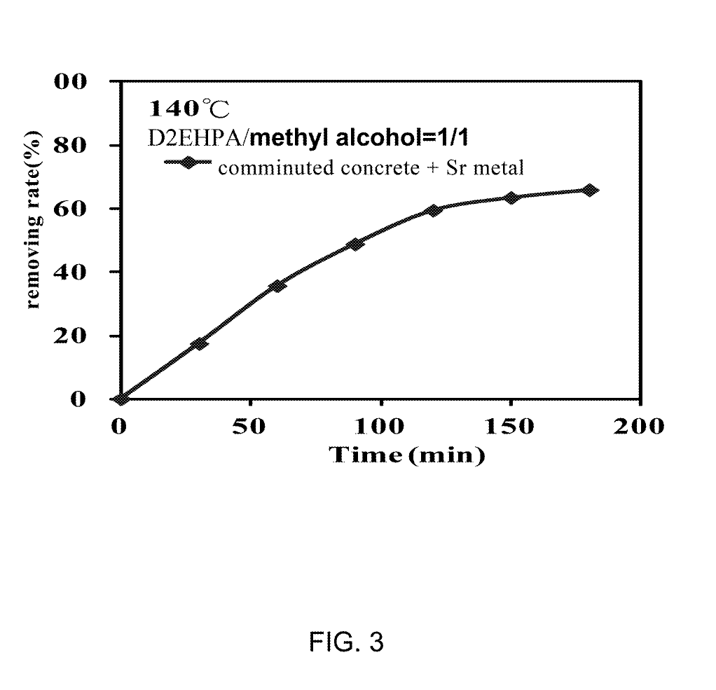

For example, in the first embodiment, the pulverized brick sample (about 280 g) is first put inside the container room 53. Under 140° C., the cosolvent (methyl alcohol), the metal chelating agent (D₂EHPA), and the supercritical flow are mixed together in a preferred weight proportion of 5.3%, 6.4%, and 88.3%. A flow speed 30 g/min. of carbon dioxide is provided. The cosolvent and metal chelating agent has a flow speed of 4.5 mL/min as designated into the container room 53. After the washing process, the pulverized brick sample is taken off, and the total metal Sr containment of the processed and non-processed pulverized brick sample is 195.9 mg and 581.4 mg, respectively. And the metal Sr decontaminated portion is found to be up to 66.3%, relating to an extraction curve as shown in FIG. 3.

(D) Sr metal experiment reduction in a pulverized concrete sample:

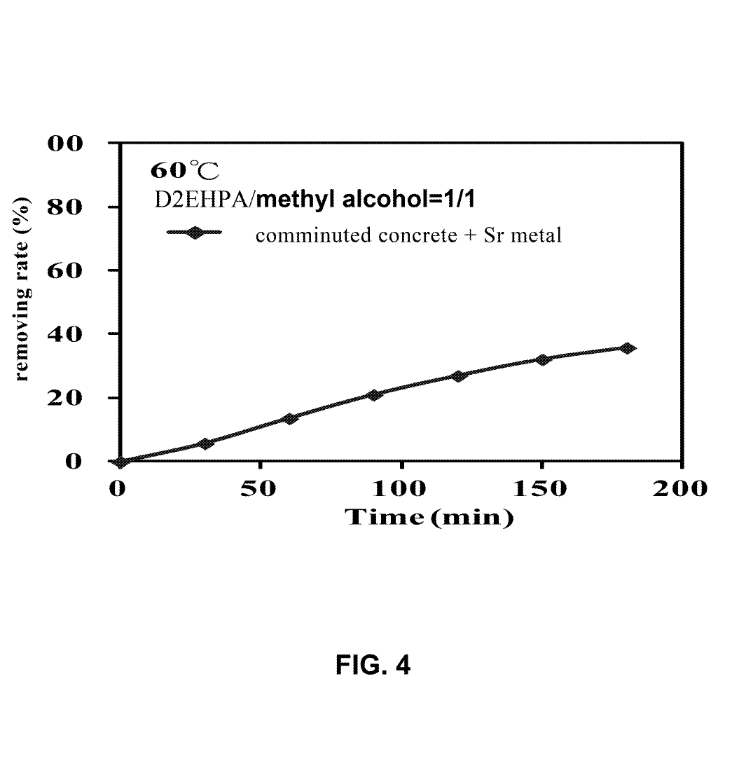

For example, in the second embodiment, the pulverized brick sample (about 370 g) is first put inside the container room 53. Under 60° C., the cosolvent (methyl alcohol), the metal chelating agent (D₂EHPA), and the supercritical flow are mixed together in a preferred weight proportion of 4.4%, 6.5% and 89.1%. A flow speed 30 g/min. of carbon dioxide is provided. The cosolvent and metal chelating agent has a flow speed of 4.5 mL/min as designated into the container room 53. After the washing process, the pulverized brick sample is taken off, and the total metal Sr containment of the processed and non-processed pulverized brick sample is 1927.1 mg and 3080.7 mg, respectively. And the metal Sr decontaminated portion is found to be up to 37.4%, relating to an extraction curve as shown in FIG. 4.

According to the first and second embodiments with respect to the brick or concrete sample, the methods (A) and (B) are examined. It is known that the decontamination agent composed of methyl alcohol, D₂EHPA, and the supercritical carbon dioxide flow does effectively decontaminate the Sr metal within the brick or concrete.

In view of the above, the present invention does have an effect of reducing the containment of the radionuclide in brick or concrete. Further, according to the containment of the radionuclide, methyl alcohol is selected as the cosolvent to enhance the effectiveness of processing the Sr metal. Accordingly, the method for decontaminating a brick or concrete does provide an improved processing effectiveness for the radionuclide in a brick or concrete. Further, in virtue of the supercritical carbon dioxide flow, the cosolvent and metal chelating agent, the radionuclide may be decontaminated under a relatively lower energy consumption with the operating temperature and pressure for both the brick and concrete, and thus effectiveness of the radionuclide decontaminating is well enhanced. Furthermore, the inventive method may consume less organic solvent and water, achieving the purpose of environment friendly and energy saving.

The above described is merely examples and preferred embodiments of the present invention, and not exemplified to intend to limit the present invention. Any modifications and changes without departing from the scope of the spirit of the present invention are deemed as within the scope of the present invention. The scope of the present invention is to be interpreted with the scope as defined in the appended claims.

1. A method for decontaminating a brick or concrete, comprises:
pulverizing the brick or the concrete by using a pulverizing device;
arranging the brick or concrete in a container room and washing the brick or concrete by using a decontamination agent in the container room, wherein the container room has a pressure of 200 to 350 Pa, and a temperature of 40 to 140° C., and the decontamination agent includes a supercritical carbon dioxide flow, a cosolvent and a metal chelating agent;
introducing a replacing fluid to the container room to replace the decontamination agent in the container room and then separating the replacing fluid with the brick or concrete; and
acid cleaning the cosolvent and the metal chelating agent by using an acidic solution.

2. The method as claimed in claim 1, wherein the metal chelating agent is selected from a the group consisting of: ithiocarbamate dibutyldithiocarbamate (BDC), dipentyldithiocarbamate (P5DC), dihexyldithiocarbamate (P6DC) or pyrrolidine-dithiocarbamate, acetylacetone (AA), trifluoroacetylacetone(TFA), hexafluoroacetyl-acetone (HFA), thenoyltrifluoroacetone (TTA) or heptafluorobutanoyl-pivaroylmethane (FOD), tributylphosphate (TBP), tributylphosphine oxide (TBPO), trioctylphosphine oxide(TOPO), triphenylphosphine oxide (TPPO)-bis(2,4,4,-trimethylpentyl) phosphinic acid (Cyanex 272), bis(2,4,4,-trimethylpentyl) dithiophosphinic acid (cyanex 301), Bis(2,4,4,-trimethylpentyl) monothiophosphinic acid (cyanex 302), and di(2-ethylhexyl) phosphoric acid (D2EHPA) or crown ether.

3. The method as claimed in claim 1, wherein the metal chelating agent is di(2-ethylhexyl) phosphoric acid.

4. The method as claimed in claim 1, wherein the cosolvent is a low-carbon alkane or alcohol, having a number of carbon of 1 to 6.

5. The method as claimed in claim 4, wherein the cosolvent is methyl alcohol or n-hexane.

6. The method as claimed in claim 1, wherein the pressure of the container room is above 74 bar.

7. The method as claimed in claim 1, wherein the pressure of the container room is 100 to 350 bar.

8. The method as claimed in claim 1, wherein the temperature of the container room is above 32° C.

9. The method as claimed in claim 1, wherein the temperature of the container room is 35° C. to 140° C.

10. The method as claimed in claim 1, wherein the cosolvent has a weight percentage of 1.0 to 20.0%, the metal chelating agent has a weight percentage of 0.1 to 9.0%, the supercritical carbon dioxide flow has a weight percentage of 79.9 to 98.9% in the decontamination agent, respectively.
